SEQUENCE LISTING (2 sequences)

SEQ ID NO: 1 - DNA, 20 bp, synthetic construct
  source 1..20: mol_type = other DNA; note = Forward primer sequence based on 16S rRNA gene for detecting Akkermansia muciniphila
ccttgcggttggcttcagat

SEQ ID NO: 2 - DNA, 20 bp, synthetic construct
  source 1..20: mol_type = other DNA; note = Reverse primer sequence based on 16S rRNA gene for detecting Akkermansia muciniphila
cagcacgtgaaggtggggac

(19) United States
(12) Patent Application Publication
(10) Pub. No.: US 2025/0288564 A1
(43) Pub. Date: Sep. 18, 2025

(54) DIHYDROBERBERINE COMPOSITION AND USE THEREOF

(71) Applicant: NANJING NUTRABUILDING BIO-TECH CO., LTD., Nanjing (CN)

(72) Inventors: Long JIANG, Nanjing (CN); Kylin LIAO, Nanjing (CN); Yunhua ZHANG, Nanjing (CN)

(21) Appl. No.: 19/066,544

(22) Filed: Feb. 28, 2025

Related U.S. Application Data
(63) Continuation of application No. PCT/CN2023/114731, filed on Aug. 24, 2023.

(30) Foreign Application Priority Data
Aug. 30, 2022 (CN) 202211048668.4

(51) Int. Cl. A61K 31/4375; A61P 1/00; A61P 31/04

(52) U.S. Cl. CPC A61K 31/4375; A61P 1/00; A61P 31/04

(57) ABSTRACT

Disclosed herein are a dihydroberberine composition and use thereof in the preparation of medicaments, nutritional products, health-care products, foods and beverages for regulating the intestinal flora. The dihydroberberine composition of the present invention can significantly increase the level of a first intestinal microbiota in a subject, while reducing the level of a second intestinal microbiota in the subject, thereby improving the intestinal health.

### CROSS REFERENCE TO RELATED APPLICATIONS

This application is a continuation application of International Patent Application No. PCT/CN2023/114731, filed on Aug. 24, 2023, which claims the priority of the Chinese patent application No. 202211048668.4, filed on Aug. 30, 2022. The contents of International Patent Application No. PCT/CN2023/114731 and Chinese patent application No. 202211048668.4 are incorporated herein by reference in their entireties.

### REFERENCE TO AN ELECTRONIC SEQUENCE LISTING

The contents of the electronic sequence listing (SequenceListing.xml; Size: 3,310 bytes; and Date of Creation: Jun. 9, 2025) is herein incorporated by reference.

### FIELD OF THE INVENTION

The invention belongs to the technical field of health-care foods and dietary supplements, and specifically relates to a dihydroberberine composition and related applications thereof in medicaments, nutritional supplements, health-care products, foods and beverages.

### BACKGROUND OF THE INVENTION

There are many long-term colonized microorganisms in human intestine, which can be divided into probiotics, harmful bacteria and conditional pathogenic bacteria according to their relationship with human health. Intestinal flora plays a very important role in human health. It has been proved that flora imbalance is related to the occurrence and development of many metabolic diseases (such as hyperlipidemia and diabetes), cancer and nervous system diseases. Among many influencing factors, diet is the key to shape and adjust the balance of flora. When the diet structure changes, it can quickly detect the changes of diet-related flora diversity, especially the intake of carbohydrates. Therefore, at present, based on different health demands, a variety of microbial regulators (such as antibacterial agents and prebiotics) or live bacteria products have been developed to maintain the body's steady state by changing the composition of the host microbiota.

The pharmacological effects of berberine on obesity, anti-inflammatory, anti-cancer, anti-obesity, anti-diabetes, anti-hyperlipidemia and heart protection have been widely studied, and under certain conditions, the composition, structure and quantity changes of intestinal flora may be improved by taking berberine.

Berberine may cause gastrointestinal discomfort such as nausea and vomiting when taken. Dihydroberberine is a metabolite of berberine in vivo, which has higher water solubility, five times higher bioavailability, longer action time, can be directly absorbed by human tissues, takes effect faster, and can relieve intestinal discomfort that may occur when berberine is taken, so it is very suitable for use in dietary supplements. Therefore, it is very important to study the composition and method of dihydroberberine to enhance intestinal health through its influence on some microbiota.

### SUMMARY OF THE INVENTION

The present invention relates to a dihydroberberine (DHB) composition and use thereof. The invention provides a method for regulating intestinal flora by dihydroberberine, in particular to regulating the levels of probiotics, harmful bacteria and conditional pathogenic bacteria in intestinal microbiota and the ratio of probiotics to harmful bacteria. Among them, the probiotics include Lactobacillus acidophilus, Akkermansia muciniphila, Faecalibacterium Prausnitzii etc., the harmful bacteria include Klebsiella pneumoniae, Desulfovibrio etc., the conditional pathogenic bacteria include Ruminococcus, Bacteroides fragilis and Enterobacter cloacae etc.

We have found that administration of a composition comprising dihydroberberine or a derivative thereof can regulate intestinal microbiota to enhance intestinal health, including increasing the level of some beneficial microbiota and decreasing the level of some harmful microbiota.

In order to achieve the above objects, the present invention provides the following technical solutions:

One aspect of the invention relates to a method for regulating or improving intestinal health in a subject, the method comprises administering a composition comprising dihydroberberine or a derivative thereof to the subject; wherein the composition comprising dihydroberberine or a derivative thereof increases the level of a first intestinal microbiota in the subject; the first intestinal microbiota is selected from one or more of the following: Lactobacillus acidophilus, Akkermansia muciniphila, Faecalibacterium Prausnitzii, Bacteroides fragilis.

In some embodiments, the method further comprises reducing the level of a second intestinal microbiota in the subject, wherein the second intestinal microbiota is Klebsiella pneumoniae, Desulfovibrio.

In some embodiments, the first intestinal microbiota is a beneficial microbiota or conditional pathogenic bacteria.

In some embodiments, the second intestinal microbiota is a harmful microbiota.

In some embodiments, the subject suffers from an intestinal disease.

In some embodiments, the composition comprising dihydroberberine is formulated into nutritional supplement, food, beverage, animal feed or medicament.

In some embodiments, the composition comprising dihydroberberine is prepared into solid preparation or liquid preparation.

In some embodiments, the method comprises administering the composition comprising dihydroberberine or a derivative thereof to the subject at a dosage of 30 to 1800 mg per day.

In some embodiments, the method comprises administering the composition comprising dihydroberberine or a derivative thereof to the subject at a dosage of 50 to 1000 mg per day.

In some embodiments, the composition comprising dihydroberberine is in the form of suppository, tablet, pill, granule, powder, film, capsule, beverage, aerosol, spirit, tincture, tonic, liquid suspension or syrup.

Another aspect of the present invention relates to the use of a composition comprising dihydroberberine in the preparation of a nutritional supplement, food, beverage, animal feed or medicament for increasing the level of a first intestinal microbiota in a subject, wherein the first intestinal microbiota is selected from one or more of the following: Lactobacillus acidophilus, Akkermansia muciniphila, Faecalibacterium Prausnitzii, Bacteroides fragilis.

In some embodiments, the use further comprises reducing the level of a second intestinal microbiota in the subject, wherein the second intestinal microbiota is Klebsiella pneumoniae, Desulfovibrio.

In some embodiments, the first intestinal microbiota is a beneficial microbiota or conditional pathogenic bacteria.

In some embodiments, the second intestinal microbiota is a harmful microbiota.

In some embodiments, the use comprises administering the composition comprising dihydroberberine or a derivative thereof to the subject at a dosage of 30 to 1800 mg per day; wherein the composition comprising dihydroberberine increases the level of the first intestinal microbiota in the subject.

In some embodiments, the use comprises administering the composition comprising dihydroberberine or a derivative thereof to the subject at a dosage of 50 to 1000 mg per day.

In some embodiments, the subject suffers from an intestinal disease.

In some embodiments, the composition comprising dihydroberberine is prepared into solid preparation or liquid preparation.

In some embodiments, the composition comprising dihydroberberine is in the form of suppository, tablet, pill, granule, powder, film, capsule, beverage, aerosol, spirit, tincture, tonic, liquid suspension or syrup.

The composition comprising dihydroberberine or a derivative thereof can increase the level of Akkermansia muciniphila, which, as a representative strain of Verrucomicrobia, accounts for more than 80% of the total number of Verrucomicrobia. This strain is colonized in the mucous layer of gastrointestinal tract, and it is one of the most abundant species in human intestinal microbiota, and its abundance is inversely proportional to the weight of mice and humans and possibilities of getting type 1 diabetes. Akkermansia muciniphila can improve the intestinal microenvironment that controls inflammation, intestinal barrier and intestinal peptide secretion. The composition comprising dihydroberberine can also increase the level of Lactobacillus acidophilus, which is the main probiotic in the small intestine, and can adjust the balance of intestinal flora, secrete antibiotic analogues to produce antagonistic effects on pathogenic bacteria, and in addition, it can produce synergistic effects with probiotics such as Bifidobacterium and restore the balance of intestinal flora. In addition, the composition comprising dihydroberberine can also increase the level of Faecalibacterium Prausnitzii. The existence of Bacteroides fragilis is beneficial to human body, but too high or too low abundance may affect health, and the composition of the invention can increase the level of Bacteroides fragilis.

The composition comprising dihydroberberine or a derivative thereof can reduce the level of Klebsiella pneumoniae, and Desulfovibrio. Among them, Klebsiella pneumoniae is widely distributed in the human intestine, with strong resistance to the external environment and strong pathogenicity. When the host's resistance decreases, it will induce symptoms such as inflammation and diarrhea. Desulfovibrio has the ability to reduce sulfate. Because the reduction product H₂S stimulates the intestine, it will lead to intestinal sensitivity and abdominal pain. Related studies have found that the abundance of Desulfovibrio in patients with intestinal diseases such as irritable bowel syndrome and ulcerative enteritis is higher than that in normal people, which also proves the harmful effect of the bacteria on intestinal health, and reducing its level is helpful to restore intestinal health.

### BRIEF DESCRIPTION OF DRAWINGS

FIGS. 1A and 1B are graphs showing changes in the relative abundance of microbiota in feces of volunteers after treatment with dihydroberberine. FIG. 1A is a graph of relative abundance changes of Lactobacillus acidophilus, Bacteroides fragilis, Akkermansia muciniphila, and Faecalibacterium Prausnitzii. FIG. 1B is a graph of relative abundance changes of Klebsiella pneumoniae and Desulfovibrio.

FIGS. 2A and 2B are graphs showing changes in the relative abundance of microbiota in feces of mice after treatment with dihydroberberine. FIG. 2A is a graph of relative abundance changes of Lactobacillus acidophilus, Bacteroides fragilis, Akkermansia muciniphila, and Faecalibacterium Prausnitzii. FIG. 2B is a graph of relative abundance changes of Klebsiella pneumoniae and Desulfovibrio.

FIG. 3 is a graph showing the change of relative abundance ratio of Bacteroides and Firmicutes in mouse feces.

FIG. 4 is a graph showing the change of absolute amount of Akkermansia muciniphila in mouse feces.

FIGS. 5A through 5C are the intestinal pathological results of three groups of mice.

### DETAILED DESCRIPTION OF THE INVENTION

Reference will now be made in detail to the embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

The present application has found that the administration of dihydroberberine can significantly increase the level of a first intestinal microbiota in a subject, while reducing the level of a second intestinal microbiota in the subject. To this end, the present invention and embodiments thereof are directed to methods using and compositions of “dihydroberberine-containing” compositions and/or “dihydroberberine-enhanced” compositions, wherein dihydroberberine-containing compositions include any composition having dihydroberberine or a dihydroberberine derivative.

As used herein, the terms “subject” or “individual” are used interchangeably to refer to any subject to whom the disclosed methods and compositions can be applied or administered. The subject may have a disease or disorder, but the subject does not need to be sick to benefit from the disclosed methods and compositions. Subjects may need to improve their intestinal and/or overall health, but they may also have overall healthy intestine and wish to maintain or further improve their intestinal and/or overall health. Therefore, any subject can take the disclosed composition or become a recipient of the disclosed method. As used herein, the term “subject” refers to animals (for example, birds, reptiles and mammals). In some embodiments, the subject can be mammals including non-primates (e.g., camels, donkeys, zebras, cows, horses, cats, dogs, rats and mice) and primates (e.g., monkeys, chimpanzees and humans). In certain embodiments, the subject may be a non-human mammal. In other embodiments, the subject may be a human.

The term “administer”, “administering”, or “administration”, as used in this invention, refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

### Composition and Administration

The present invention relates to a method for increasing the level of a first intestinal microbiota and also reducing the level of a second intestinal microbiota in a subject by administering a dihydroberberine-containing composition to the subject at a certain dose.

Depending on the particular dihydroberberine-containing formulation and form, contemplated methods include an administration of at least 30 mg/day of dihydroberberine or a dihydroberberine derivative. This amount per day may be administered at once or in multiple doses. Typically, an effective amount of dihydroberberine or a dihydroberberine derivative in the dihydroberberine-containing composition to be administered at once or in multiple doses per day is of or between 30 milligrams (mg) to 1800 mg. One or more doses may be administered once a day for any period of time. For example, the effective dose may be administered each day for one day, a few days, multiple days, or on a daily basis indefinitely. More typically, the amount of dihydroberberine or a dihydroberberine derivative to be administered each day is 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg. More typically, the amount of dihydroberberine or a dihydroberberine derivative to be administered each day is 30 mg to 1000 mg. Most typically, the amount of dihydroberberine or a dihydroberberine derivative to be administered each day is 200 mg.

Multiple forms and formulations of the composition comprising dihydroberberine or a derivative thereof are contemplated for increasing the level of the first intestinal microbiota and also decreasing the level of the second intestinal microbiota in the mammal. Compositions will be formulated as a nutritional or dietary supplement, in a (medical) food item, in animal feed, or as a pharmaceutical composition in liquid or solid form comprising the composition comprising dihydroberberine or a derivative thereof, and may optionally also include a nutritionally or pharmaceutically acceptable carrier. For example, where the composition is in solid form, the compositions may be formulated as a snack bar, yogurt, lozenge, tablet, or capsule, or may be coated onto cereal products, included in baked goods. On the other hand, where the supplement is in liquid form, the compositions may be formulated as a tincture, soft gel capsule, liquid capsule, syrup, carbonated drink, a brewed beverage (e.g., as coffee or tea), a juice, an energy drink, a sports drink, or flavored water. While nutritional and pharmaceutical compositions for human use are especially contemplated, it should be appreciated that the composition and formulation comprising dihydroberberine or a derivative thereof may also be employed for veterinary use (e.g., use in animal feed for domestic companion animals (‘pets’) or in animal feed for farm animals. In further contemplated aspects, the composition comprising dihydroberberine or a derivative thereof may also be provided as a bulk product (e.g., in quantities of equal or greater than 100 g, equal or greater than 1,000 g, or equal or greater than 10 kg) for use in production of the nutritional supplement, a (medical) food item, animal feed, or pharmaceutical product.

The composition comprising dihydroberberine or a derivative thereof and its formulation of the invention can be applied to dietary supplements for preventing cardiovascular diseases, reducing blood fat and blood sugar, and resisting inflammation, bacteria and infection.

Composition comprising dihydroberberine or a derivative thereof may also be added to a food item comprising microbiota. The inventors contemplate a method for increasing the probiotic benefits of the food item comprising microbiota strains by combining or adding a composition comprising dihydroberberine or a derivative thereof to the food item. In this way, the diydroberberine or diydroberberine derivative may be processed by the consumer concomitantly with the microbiota strain in the food item thereby enhancing the probiotic effects. As used herein, a food item includes any solid or liquid form of food or drink that may be consumed or ingested. As disclosed herein, the amount of the diydroberberine-containing composition to be added to the food item comprising a Bifidobacterium strain may vary depending on the kind and form of the food item. Typically, the amount of the diydroberberine-containing composition would be determined on an approximate per serving basis of the food item. More typically, the amount of the diydroberberine-containing composition would be of or between 50 mg to 1,800 mg of diydroberberine or a diydroberberine derivative per serving of the food item. Most typically, the amount of the diydroberberine-containing composition would be 100 mg, 200 mg, 300 mg, 400 mg, 500 mg, 600 mg, 700 mg, 800 mg, 900 mg, 1000 mg of diydroberberine or a diydroberberine derivative per serving of the food item.

Additional aspects of the inventive subject matter may include adding the diydroberberine-containing composition and/or a diydroberberine-enhanced composition to a food item as disclosed herein. In a particular example, yogurt or a yogurt drink is a product which already contains bacteria species, such as Lactobacillus bulgaricus and Streptococcus thermophilus, which are used for fermentation. Accordingly, it is contemplated that yogurt may be supplemented with a diydroberberine-containing composition and/or a diydroberberine-enhanced Bifidobacterium strain for a synbiotic product for gut health.

### Combination Therapy

To further enhance the gut health of the subject, aspects of the contemplated methods of administering a diydroberberine-containing composition include administering at least one probiotic microorganism concomitantly or subsequent to the administration of the diydroberberine-containing composition. Any suitable probiotic or multiple probiotics may be combined with the diydroberberine-containing composition.

The presently disclosed compositions comprising diydroberberine or a derivative thereof (e.g., in liquid or solid form or as a nutritional supplement) or diydroberberine-enhanced compositions may be combined with one or two of the prebiotics of arabinoxylan (AX), an arabinoxylan oligosaccharide (AXOS), xylooligosaccharide (XOS), fructooligosaccharide (FOS), galactooligosaccharide (GOS), inulin, and/or pectin.

Any of the presently disclosed diydroberberine-containing compositions (e.g., in liquid or solid form or as a nutritional supplement) or diydroberberine-enhanced compositions may be combined with a health additive to increase the breadth of the health benefits. These health additives may be added in any combination with the diydroberberine-containing compositions or diydroberberine-enhanced compositions depending on the form of the composition as well as consideration for the desired health effect and product cost. Exemplary additives that may be combined with diydroberberine-containing compositions or diydroberberine-enhanced compositions include superoxide dismutase (SOD), compositions comprising activators of SOD, foods or extracts thereof comprising bioavailable SOD (e.g., sprouted wheat, wheatgrass, encapsulated (lipid and/or protein encapsulation) cantaloupe, rye, barley, barley grass, broccoli sprouts, kale, brussel sprouts, and curcumin (e.g., turmeric), copper I (Cu I), selenium (Se), fulvic acid, foods or extracts thereof comprising fulvic acid (e.g., potatoes, radishes, beets, carrots, root vegetables, blackstrap molasses, and shilajit), Co enzyme Q₁₀ (ubiquinone), or pyrroloquinoline quinone (PQQ).

### EXAMPLES

Intestinal flora mainly includes four phyla, namely Bacteroidetes (28%), Firmicutes (64%), Proteobacteria (8%) and Actinobacteria (3%), which cover 99% of intestinal bacteria, in addition, bacteria of Verrucomicrobia, Fusobacteria, Cyanobacteria and Spirochaetes also exist in human intestine.

In the present invention, Bacteroides fragilis is the representative of Bacteroidetes, and its practical application includes but is not limited to the common strains of Bacteroides vulgatus, Bacteroides thetaiotaomicron, Bacteroides distasonis, ect. in this phylum. Lactobacillus acidophilus and Faecalibacterium Prausnitzii are the representatives of Firmicutes, and their practical applications include but are not limited to the common strains of Bacillus, Lactobacillus and Staphylococcus in this phylum. Klebsiella pneumoniae and Desulfovibrio are representatives of Proteobacteria, and their practical applications include but are not limited to the strains of Salmonella, Desulfovibrio, Desulfotomacvlum and Vibrio, which are common in this phylum. The representative genera of Actinobacteria include Bifidobacterium, Micrococcus, Actinomyces, etc., and their practical applications include but are not limited to these.

### Example 1

Experimental method: Six volunteers (three males and three females) were recruited to obtain relevant health data, and it was confirmed that they had not taken antibiotics or probiotics in the previous three months, and were healthy and had no chronic diseases. A 3-week experiment was designed. Volunteers had 30 minutes of moderate-intensity exercise every day, and ensured regular work and rest, reasonable diet, and no smoking or drinking. They were administered 200 mg of dihydroberberine (DHB) every day, and fecal samples were collected on days 0, 7, 14 and 21 of the experiment, and the corresponding bacterial species were analyzed.

Before the experiment, fecal samples of each volunteer were collected, and species information was obtained through genomic DNA extraction, PCR amplification of 16s RNA and database comparison.

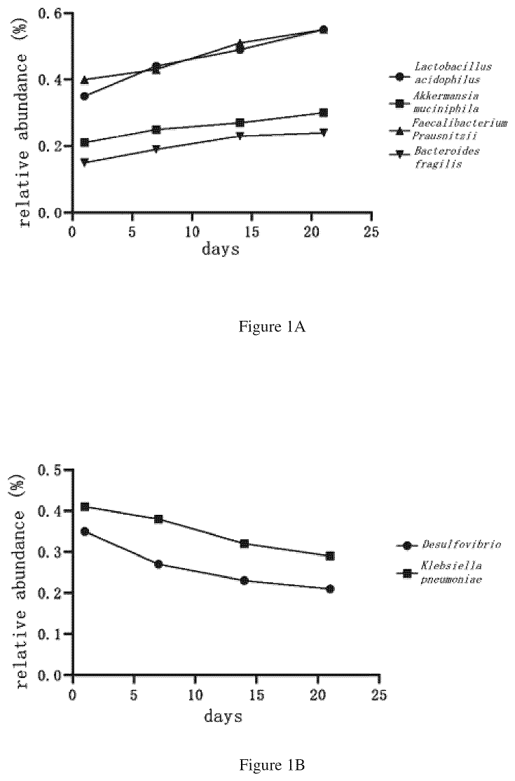

The results are shown in FIG. 1. FIG. 1A shows that the relative abundance levels of Lactobacillus acidophilus, Akkermansia muciniphila, Faecalibacterium Prausnitzii and Bacteroides fragilis increased by 0.20%, 0.09%, 0.15% and 0.11% respectively after dihydroberberine treatment. FIG. 1B shows that the relative abundance levels of Klebsiella pneumoniae and Desulfovibrio decreased by 0.12% and 0.14% respectively after dihydroberberine treatment.

### Example 2

Experimental method: After a week of adaptive feeding, mice were randomly divided into four groups according to sex and weight: group 1, control group, n=10; group 2, normal+DHB group, n=10; group 3, high-fat-high-sugar group, n=10; and group 4, high-fat-high-sugar+DHB group, n=10. High-fat-high-sugar group (group 3) and high-fat-high-sugar+DHB group (group 4) were given high-fat and high-sugar feed (containing 35% fat, 18% protein and 47% carbohydrate), while the control group and normal+DHB group were given normal feed. After eight weeks, blood was taken from the tail vein to detect blood sugar, and the groups 3 and 4 were given high-fat and high-sugar feed, while the control group and normal+DHB group (groups 1 and 2) were given basic feed until the end of the experiment. Then, groups 2 and 4 were administered DHB at 100 mg/kg (dissolved in physiological saline) for 4 weeks, and fecal samples were taken on days 0, 14 and 28. The maximum dose of DHB refers to the usual dose of daily health care, and the dosage is set to 200 mg/kg. The specific gavage scheme is: dissolve DHB in water and gavage once a day according to the weight of mice, while the control group gavage with the same amount of pure water.

Analysis of the relative abundance of intestinal flora: Four samples of mouse feces were randomly taken from each group. The samples were first extracted for DNA, 16S rDNA was amplified by PCR, and then sequenced. After completing the classification of data species, the relative abundance of each species was analyzed.

Abundance ratio of Bacteroides and Firmicutes:Bacteroides is the representative of intestinal probiotics and Firmicutes is the representative of intestinal harmful bacteria. The higher the ratio, the healthier the intestinal flora is.

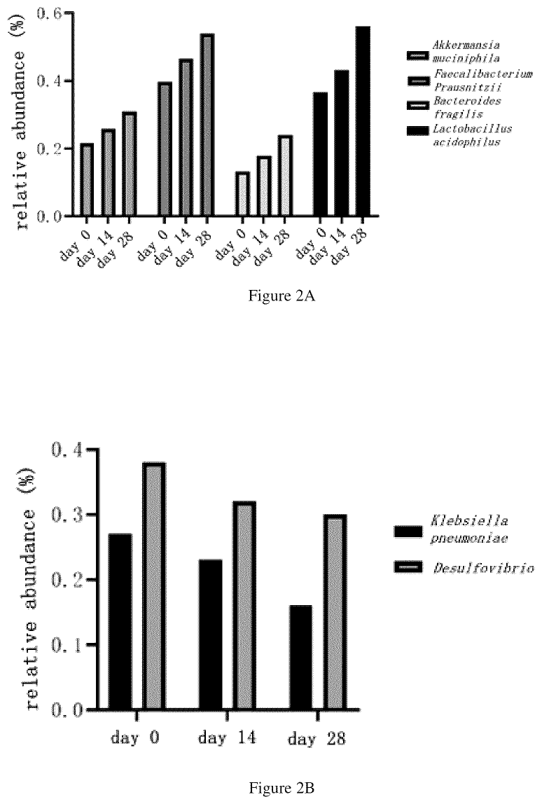

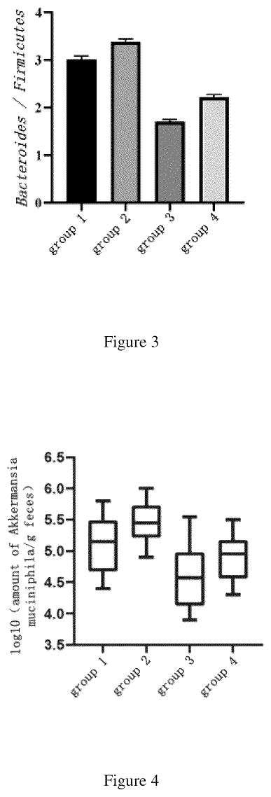

Determination of absolute amount of Akkermansia muciniphila in mouse feces: The colon feces of mice were collected, the absolute amount of Akkermansia mucinipha in each gram of feces was detected, and the influence of the composition of the invention on the absolute amount of Akkermansia mucinipha in the intestines of normal and high-fat-high-sugar mice was analyzed. 1) Isolation and extraction of DNA from feces samples: the newly collected mouse feces samples or feces samples stored at −80° C. immediately after collection were used to extract genomic DNA with Tiangen feces genome extraction kit. 2) qPCR detection: ABI 7500Fast real-time PCR system was used for detection. The primer sequence based on 16S rRNA gene for detecting Akkermansia muciniphila is as follows: F-5′ CCTTGCGGTTGGCTTCAGAT 3′ (SEQ ID NO: 1) and R-5′ CAGCACGTGAAGGTGGGGAC 3′ (SEQ ID NO: 2). The cycle threshold (Ct) of each sample was compared with the standard curve (repeated three times) obtained by diluting the standard genomic DNA (5 times continuous dilution). The data is expressed as the logarithm of the number of bacteria per gram of feces (Log 10), and the results are shown in FIGS. 2-4. Compared with normal mice, the flora level of high-fat-high-sugar mice has changed significantly, while DHB treatment can make the flora level of high-fat-high-sugar mice close to that of normal mice. As shown in FIG. 2A, the relative abundance levels of Lactobacillus acidophilus, Akkermansia muciniphila, Faecalibacterium Prausnitzii and Bacteroides fragilis increased by 0.19%, 0.09%, 0.14%, and 0.10%, respectively. As shown in FIG. 2B, after dihydroberberine treatment, the relative abundance levels of Klebsiella pneumoniae and Desulfovibrio decreased by 0.11% and 0.08% respectively. As shown in FIG. 3, DHB can significantly increase the ratio of Bacteroides to Firmicutes in groups 2 and 4, with group 2 increasing by 0.36 compared with group 1 and group 4 increasing by 0.51 compared with group 3, indicating that DHB can up-regulate the level of intestinal probiotics. As shown in FIG. 4, after administration of DHB, the logarithm number of Akkermansia muciniphila per gram of feces increased significantly, group 2 increased by 0.65 order of magnitude compared with group 1, and group 4 increased by 0.55 order of magnitude compared with group 3.

### Example 3

Experimental method: Thirty 4-6-week-old mice, 15 males and 15 females, were selected for adaptive feeding for 7 days in advance. They were randomly divided into control group (CON), model group (DSS) and treatment group (DSS+DHB), with 10 mice in each group. The model group and the treatment group were gavaged with 3% dextran sodium sulfate (DSS) for 9 days to establish an acute colitis model, while the control group was gavaged with the same amount of normal saline every day. The weight, feces and rectal bleeding of mice were recorded every day, and the disease activity index (DAI) was used to evaluate the condition of colitis. The DAI value was obtained by adding all the scores and dividing them by three. The scoring standard is shown in Table 1. Mice in the treatment group were given DHB at a daily dose of 50 mg/kg. On the 10th day, blood was taken from the orbit of each mouse, and then it was killed. Colon tissues were collected, fixed in 4% paraformaldehyde solution and stored at −80° C. for subsequent analysis. The pathological damage of colon tissue was examined by hematoxylin-eosin staining and Alxin blue staining.

After extracting total RNA from colon tissue with Trizol reagent, Reveraid First Strand cDNA synthesis kit was operated according to the instructions to realize cDNA synthesis. Finally, real-time quantitative PCR analysis was carried out with SYBR Green qPCR Master Mix kit. The mRNA expression was normalized to GAPDH value, and the amplification level was calculated by 2-ΔΔCt method.

TABLE 1
DAI scoring standard

| Weight loss (%) | Feces | Bleeding | Score |
|---|---|---|---|
| <1 | normal | none | 0 |
| 1-5 | soft and lumpy | + | 1 |
| 5-10 | wet and soft | ++ | 2 |
| 10-15 | loose | +++ | 3 |
| >15 | diarrhea-like | massive | 4 |

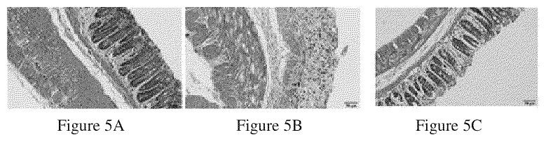

The results showed that the DAI score of the model group (DSS) mice was close to 3, while the DAI score of the treatment group (DSS+DHB) was about 1.1, which was much lower than that of the model group. The pathological changes of intestinal tract are shown in FIGS. 5A-5C, which are the intestinal structure diagrams of the control group (CON), the model group (DSS) and the treatment group (DSS+DHB) respectively. As for the intestinal structure, the epithelial structure of the model group (DSS) mice has changed compared with the normal group, showing an irregular shape. The treatment group (DSS+DHB) significantly improved the integrity of crypt structure after administration of DHB, and goblet cells were normally distributed and arranged, indicating that DHB can alleviate intestinal inflammation.

In the present invention, after dihydroberberine treatment, the content of other probiotics can be increased, such as Lactobacillus (including but not limited to Lactobacillus bulgaricus, Lactobacillus acidophilus, etc.), Bifidobacterium (including but not limited to Bifidobacterium thermophilus, Bifidobacterium lactis, etc.), Christensenellaceae, Streptococcus thermophilus, Clostridium butyricum, etc. Besides, the content of other harmful bacteria decreased, such as Proteus mirabilis, some Clostridium (such as Clostridium perfringens), Pseudomonas aeruginosa, enterotoxic Escherichia coli, Staphylococcus aureus and other intestinal pathogenic bacteria. This makes the level of these intestinal microbiota close to the normal level, which is very beneficial to maintain intestinal health.

The foregoing is only the preferred examples of the present invention, and is not intended to limit the present invention. Any person skilled in this field can make many changes, modifications, substitutions and variations on these examples without departing from the principles and purposes of the present invention. The scope of the invention is defined by the claims and their equivalents.

What is claimed is:

1. A method for regulating or improving intestinal health in a subject, wherein the method comprises administering a composition comprising dihydroberberine or a derivative thereof to the subject; wherein the composition comprising dihydroberberine or a derivative thereof increases a level of a first intestinal microbiota in the subject; the first intestinal microbiota is selected from one or more of the following: Lactobacillus acidophilus, Akkermansia muciniphila, Faecalibacterium Prausnitzii, Bacteroides fragilis.

2. The method of claim 1, wherein the method further comprises reducing a level of a second intestinal microbiota in the subject, wherein the second intestinal microbiota is Klebsiella pneumoniae or Desulfovibrio.

3. The method of claim 1, wherein the first intestinal microbiota is a beneficial microbiota or conditional pathogenic bacteria.

4. The method of claim 2, wherein the second intestinal microbiota is a harmful microbiota.

5. The method of claim 1, wherein the composition comprising dihydroberberine or a derivative thereof is formulated into a nutritional supplement, food, beverage, animal feed or medicament.

6. The method of claim 1, wherein the composition comprising dihydroberberine or a derivative thereof is prepared into a solid preparation or liquid preparation.

7. The method of claim 1, wherein the composition comprising dihydroberberine or a derivative thereof is administered to the subject at a dosage of 30 to 1800 mg per day.

8. The method of claim 1, wherein the composition comprising dihydroberberine or a derivative thereof is in a form of a suppository, tablet, pill, granule, powder, film, capsule, beverage, aerosol, spirit, tincture, tonic, liquid suspension or syrup.

9. The method of claim 1, wherein the composition comprising dihydroberberine or a derivative thereof is administered to the subject at a dosage of 50 to 1000 mg per day.
